(19) United States
(12) Patent Application Publication
(10) Pub. No.: US 2018/0333308 A1
(43) Pub. Date: Nov. 22, 2018

(54) Sponge for Gynecological Disease Treatment and Menstrual Sanitation

(71) Applicant: Beijing Yingjia Medical Material Co., Ltd., Beijing (CN)

(72) Inventor: Tieqiao ZHU, Beijing (CN)

(73) Assignee: Beijing Yingjia Medical Material Co., Ltd., Beijing (CN)

(21) Appl. No.: 15/771,419

(22) PCT Filed: Jul. 20, 2016
(86) PCT No.: PCT/CN2016/090676

§ 371 Date: Apr. 27, 2018

(30) Foreign Application Priority Data
Jun. 23, 2016 (CN) 201620631023.7

(51) Int. Cl. A61F 13/20; A61F 13/34; A61L 15/24; A61L 15/20; A61L 15/44; A61L 15/42; A61L 15/50

(52) U.S. Cl. CPC A61F 13/2074; A61F 13/34; A61F 13/2034; A61L 15/24; A61F 2013/15081; A61L 15/44; A61L 15/425; A61L 15/50; A61L 15/20

(57) ABSTRACT

A menstrual sanitary sponge for gynecological disease treatment, comprising a sponge body. The shape of the sponge body is cylindrical. The top of the sponge body is hemispherical. An annular, spiral, hemispherical, or hook-shaped groove is provided on the surface of the sponge body. The sponge body is a compressed medical polyvinyl alcohol sponge.

### TECHNICAL FIELD

The present disclosure relates to the technical field of gynecological disease treatment, and particularly to a sponge for gynecological disease treatment and menstrual hygiene.

### BACKGROUND ART

With regard to common gynecological diseases such as cervicitis, vaginitis and pelvic inflammation, due to the restriction of physiological positions thereof, many medicines fail to reach the lesion. Even if the medicine is applied to the lesion site through complex operations, due to factors such as movement and underwear friction, and mobility of liquid medicine, as well as dilution or discharge of the medicine caused by secretions of the human body, the medicine is difficult to be retained, and thus the medicine has to be applied again, which complicates the treatment process and prolongs the treatment period. In this case, the normal life of patients is affected. Moreover, since the medicine cannot be continuously and directly applied the lesion site, it is possible to delay the condition of illness and aggravate the patients' pains.

There are many kinds of menstrual sanitary materials for women in the market, however, these materials can hardly achieve non-leakage due to differences among individuals' physiological status. Moreover, a portion of shedding, blood clots etc. may leave behind in vivo for a longer period of time, which may further cause adverse effects to the health of human body.

Currently, the medical diagnosis of gynecological diseases is quite accurate, but there is an urgent need for a gynecological disease treatment device to enable the medicine to directly reach the lesion site in the vagina, and to accurately and continuously perform an effective treatment for the lesion site.

### SUMMARY OF THE DISCLOSURE

An object of the present disclosure is to provide a sponge for gynecological disease treatment and menstrual hygiene, so as to solve the problems in the prior art that medicines can hardly directly reach a lesion site in the body of women, that continuous and stable administration cannot be carried out for the lesion site, and that the resulting treatment effect is not ideal.

In order to achieve the above object, the following technical solutions are adopted in the present disclosure.

A sponge for gynecological disease treatment and menstrual hygiene provided in embodiments of the present disclosure includes a sponge body. The sponge body is in a cylindrical shape, and a top portion of the sponge body is hemispherical. A surface of the sponge body is provided with annular, spiral, hemispherical or hook-shaped grooves. The sponge body is a compressed medical polyvinyl alcohol sponge.

Furthermore, a coating is provided on a surface of the hemispherical top portion of the sponge body. The coating is a medical vaseline or antibiotic ointment coating. The technical effect of this technical solution is as follows: since the vaginal wall is not smooth, and the surface of the hemispherical top portion of the sponge body is provided with a medical vaseline or antibiotic ointment coating which can play a role of lubrication and anti-inflammation, facilitate the placement of the sponge body, and prevent the vaginal wall from being hurt and infected by friction.

Furthermore, the coating extends downwards from the hemispherical top portion of the sponge body, and covers ¼ to ½ of a total length of the sponge body. The technical effect of this technical solution is as follows: the coating covering ¼ to ½ of the sponge body makes the placement of the sponge body more successful.

Furthermore, a pulling thread is provided at bottom of the sponge body. The technical effect of this technical solution is as follows: when the sponge body needs to be taken out, the pulling thread can be dragged to slowly pull the sponge body, and the sponge body can also be pulled out with tweezers.

Furthermore, a diameter of the sponge body is 10-20 mm, and a length of the sponge body is 40-80 mm. The technical effect of this technical solution is as follows: volume of the sponge body is relatively small, and thus it is easier to place the sponge body in the vagina.

Furthermore, the diameter of the sponge body after swelling is 20-40 mm, and a length of the sponge body after swelling is 90-130 mm. The technical effect of this technical solution is as follows: generally, the diameter of the vagina of the human body can be expanded to 20-40 mm, and the length of the vagina of the human body can be expanded to 90-130 mm, in view of which the dimension of the swelled sponge body comply with the actual condition of the human body, and can expand the vagina, so that the vagina is under a uniform pressure, and the treatment effect is better.

Furthermore, strength of the sponge body is designed to be greater than 1 kg/cm². The technical effect of this technical solution is as follows: when the sponge body, after being used, is pulled out from the vagina through the pulling thread or tweezers, the sponge body may be broken in the vagina if the operation is improper, such situation is relatively hard to handle, therefore, the sponge body needs to have certain strength. The strength of the present sponge body is designed to be greater than 1 kg/cm², which can meet requirements of clinical use.

Furthermore, a breaking elongation of the sponge body is greater than 100%. The technical effect of this technical solution is as follows: the breaking elongation of the present sponge body is greater than 100%, so that the elasticity of the present sponge body can meet requirements of clinical use.

Furthermore, the sponge body is a medical polyvinyl alcohol sponge processed by cobalt-60 irradiation sterilization. The technical effect of this technical solution is as follows: the present sponge body is processed by cobalt-60 irradiation sterilization, so that the sterilization effect is significant, and requirements of clinical use can be met.

Furthermore, the sponge body is a medical polyvinyl alcohol sponge packaged through double seal. The technical effect of this technical solution is as follows: the present sponge body is packaged through double seal, so that an aseptic condition can be ensured, and requirements of storage and clinical use can be met.

The sponge for gynecological disease treatment and menstrual hygiene provided in embodiments of the present disclosure includes the cylindrical sponge body. The sponge body is a compressed medical polyvinyl alcohol sponge. The medical polyvinyl alcohol sponge has extremely good elasticity, can be well adapted to a surface of wound. After absorbing of a liquid, the medical polyvinyl alcohol sponge swells according to the body's anatomical form, and thus plays a role in supporting tissues; furthermore, a pressure is uniformly applied on the surface of wound, and the cured body tissues can maintain the original form. Moreover, the sponge body has excellent absorbability of liquid, and can absorb aqueous, oily or emulsifying liquid remarkably well. After the medicinal liquid is absorbed, due to the effect of concentration difference, the medicine will slowly spread to the lesion site, thereby playing a role of local sustained-release delivery. Meanwhile, the sponge body has good biocompatibility as well, so that a surface of wound will not generate scar if the sponge body is placed into the vagina after operation. The present sponge for gynecological disease treatment and menstrual hygiene also makes it possible to accurately locate the surface of wound, feed the medicine specifically to the lesion position in the body, producing a significant treatment effect. Moreover, when the sponge body is placed in and out from the vagina, since the coating contributes to lubrication and anti-inflammation, the vagina can be prevented from being injured to a great extent, thus reducing the patients' pains. The sponge body also can be used as a menstrual hygiene product which has a well leakage-proof effect. When the sponge body is taken out, the solid wastes such as endometrium shedding and blood clots can be carried out from the body, which is equivalent to cleaning the vagina. Such cleaning is convenient to use, hygienic and thus is suitable for popularization and application.

### BRIEF DESCRIPTION OF DRAWINGS

In order to more clearly illustrate technical solutions of embodiments of the present disclosure or the prior art, figures which are needed to be used in the description of the embodiments or the prior art will be introduced briefly below. Obviously, the figures in the following description are for some embodiments of the present disclosure. A person ordinarily skilled in the art could still obtain other figures according to these figures, without paying inventive efforts.

FIG. 1 is a structural schematic diagram of a sponge for gynecological disease treatment and menstrual hygiene provided in a first embodiment of the present disclosure, with the sponge swelled after absorbing liquid;

FIG. 2 is a structural schematic diagram of a sponge for gynecological disease treatment and menstrual hygiene provided in a second embodiment of the present disclosure, with the sponge swelled after absorbing liquid;

FIG. 3 is a structural schematic diagram of a sponge for gynecological disease treatment and menstrual hygiene provided in a third embodiment of the present disclosure, with the sponge swelled after absorbing liquid;

FIG. 4 is a structural schematic diagram of partial cross-section of a sponge for gynecological disease treatment and menstrual hygiene provided in a fourth embodiment of the present disclosure, with sponge swelled after absorbing liquid.

### REFERENCE SIGNS

sponge body 1, top portion 2, groove 3, pulling thread 4.

### DETAILED DESCRIPTION OF EMBODIMENTS

The technical solutions of the present disclosure will be described clearly and completely blow in conjunction with figures. Apparently, the described embodiments are some but not all of embodiments of the present disclosure. All the other embodiments obtained by a person ordinarily skilled in the art without paying inventive effort, based on the embodiments of the present disclosure, shall fall within the scope of protection of the present disclosure.

In the description of the present disclosure, it should be noted that orientational or positional relationships indicated by terms such as “center”, “upper”, “lower”, “left”, “right”, “vertical”, “horizontal”, “inner” and “outer” are based on orientational or positional relationships as shown in the figures, and are merely for facilitating the description of the present disclosure and simplification of the description, rather than indicating or implying that related devices or elements have to be in the specific orientation or configured and operated in specific orientation, and therefore, should not be construed as limiting the present disclosure. Besides, terms such as “first”, “second” and “third” are merely for descriptive purpose, and should not be construed as indicating or implying relative importance.

In the description of the present disclosure, it should be indicated that unless otherwise specified and defined, terms “mount”, “join” and “connect” should be construed in a broad sense. For example, the connection may be a fixed connection, a detachable connection, or an integrated connection; it may be a mechanical connection or an electrical connection; or it may be a direct connection or an indirect connection through an intermediate medium; or it may also be an inner communication between two elements. For a person ordinarily skilled in the art, specific meanings of the above-mentioned terms in the present disclosure can be understood according to specific circumstances.

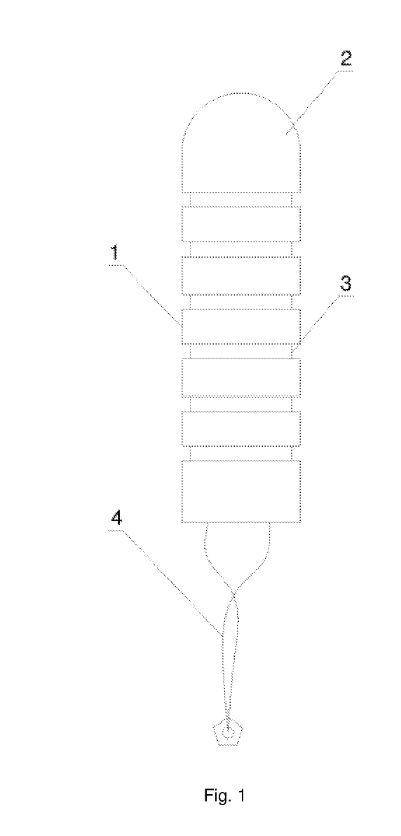

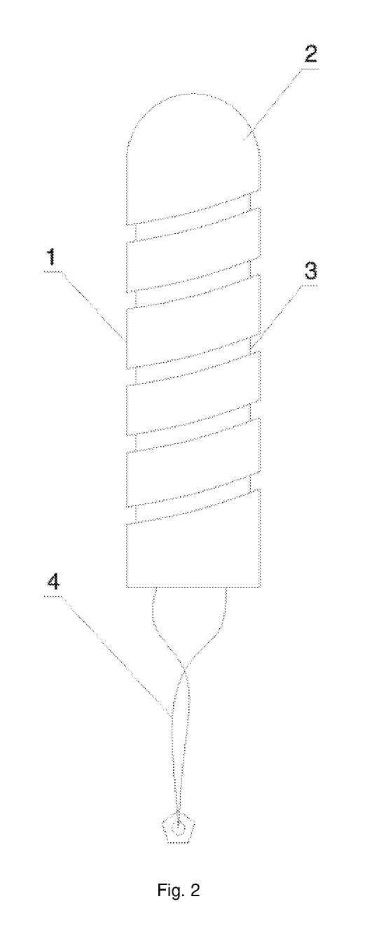

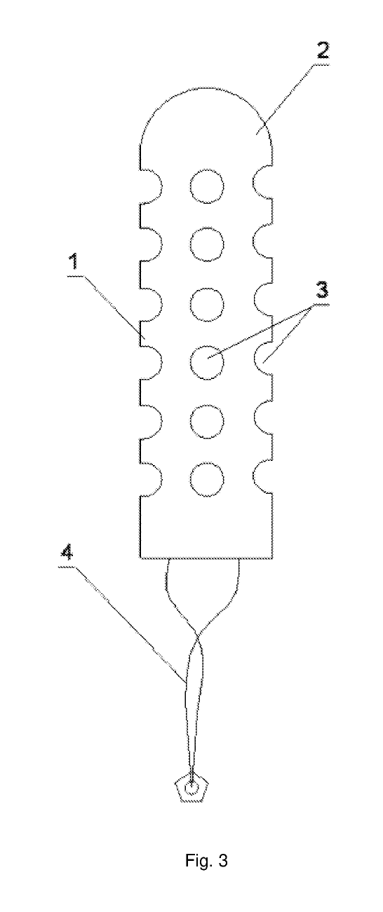

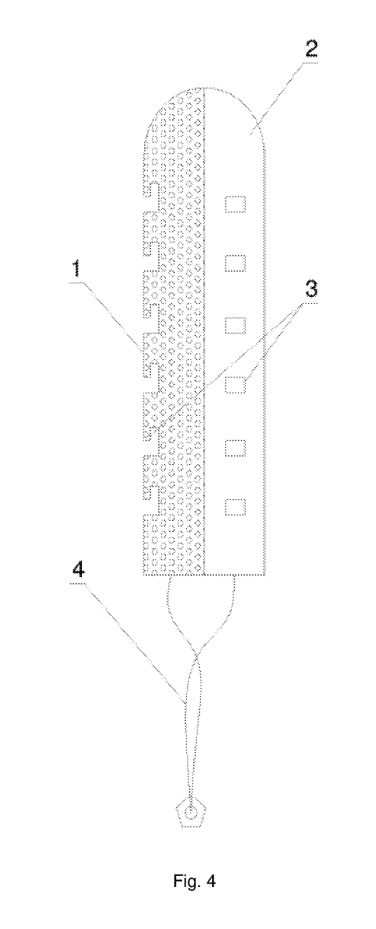

FIG. 1 is a structural schematic diagram of a sponge for gynecological disease treatment and menstrual hygiene provided in a first embodiment of the present disclosure, with the sponge swelled after absorbing liquid; FIG. 2 is a structural schematic diagram of a sponge for gynecological disease treatment and menstrual hygiene provided in a second embodiment of the present disclosure, with the sponge swelled after absorbing liquid; FIG. 3 is a structural schematic diagram of a sponge for gynecological disease treatment and menstrual hygiene provided in a third embodiment of the present disclosure, with the sponge swelled after absorbing liquid; FIG. 4 is a structural schematic diagram of partial cross-section of a sponge for gynecological disease treatment and menstrual hygiene provided in a fourth embodiment of the present disclosure, with the sponge swelled after absorbing liquid.

### First Embodiment

In an optional solution of the present embodiment, as shown in FIG. 1, a sponge for gynecological disease treatment and menstrual hygiene provided in the present embodiment includes a sponge body 1. The sponge body 1 is in cylindrical shape, and a top portion 2 of the sponge body is hemispherical. A surface of the sponge body 1 is provided with annular grooves 3. The sponge body 1 is a compressed medical polyvinyl alcohol sponge. Currently, the medical polyvinyl alcohol sponge has not yet been used for gynecological disease treatment in the medical field. The conventional treatment process is complicated and has a long treatment cycle, which would affect the normal life of the patient, therefore the patients suffer from deep pains. In the present embodiment, the sponge body 1 is in a cylindrical shape and the top portion 2 thereof is hemispherical, which makes it is easy to place the sponge body into the vagina. The sponge body 1 is a compressed medical polyvinyl alcohol sponge. The medical polyvinyl alcohol sponge has extremely good elasticity, can be well positioned at and adapted to a surface of wound, and can apply a pressure uniformly to the surface of wound after swelling, due to absorption of liquid, according to the body's anatomical form. In this case, the cured body tissues can maintain the original form. Additionally, the medical polyvinyl alcohol sponge can well absorb the medicinal liquid, and can thus play a role of local sustained-release delivery. Therefore, the technical problems, such as the inaccurate location on the surface of wound in the vagina, non-uniform pressure, and poor treatment effect, are solved. Clinically, the medicinal liquid is injected into the sponge body 1, and then the sponge body with the medicinal liquid is placed against the lesion in the vagina. In this way, the medicine can be stably administered at a fixed point in large dose, and thus, the medicine can directly enter the lesion to kill pathological cells. With the sponge for gynecological disease treatment and menstrual hygiene provided in the present embodiment, the medicine has not to be administered by entering the digestive tract and the blood circulation system, and can be directly applied to the pathological cells in the vagina, so that the pathological cells are easier to be killed and the side effects of the medicine on the human body are significantly decreased, which provides more choices on the medicines for the doctors. The sponge for gynecological disease treatment and menstrual hygiene provided in the present embodiment may be also used as a menstrual hygiene product which has a well leakage-proof effect. When the sponge body is taken out, the annular grooves 3 may carry, solid wastes such as endometrium shedding and blood clots, out from the body, which is equivalent to cleaning the vagina. Such sponge is convenient to use, safety and hygienic.

Furthermore, in an optional solution of the present embodiment, a coating is provided on the surface of the hemispherical top portion 2 of the sponge body 1. The coating is a medical vaseline or antibiotic ointment coating. In the present embodiment, this coating can play a role of lubrication and anti-inflammation, and prevent the vaginal wall from being hurt and infected by friction.

Furthermore, in an optional solution of the present embodiment, the coating extends downwards from the hemispherical top portion 2 of the sponge body 1, and covers ⅓ of a total length of the sponge body 1. In the present embodiment, the coating covers ⅓ of the total length of the sponge body 1, so that the sponge body 1 can be more smoothly and conveniently placed into the vagina.

Furthermore, in an optional solution of the present embodiment, a pulling thread 4 is provided at bottom of the sponge body 1. In the present embodiment, when the sponge body 1 needs to be taken out of the vagina, the pulling thread 4 can be dragged to slowly pull the sponge body 1 out; the sponge body 1 can also be slowly pulled out with the help of tweezers.

Furthermore, in an optional solution of the present embodiment, a diameter of the sponge body 1 is preferably 15 mm, and a length of the sponge body is preferably 60 mm.

Furthermore, in an optional solution of the present embodiment, a diameter of the sponge body 1 after swelling is preferably 30 mm, and a length of the sponge body after swelling preferably 110 mm. In the present embodiment, the preferable dimension of the swelled sponge body 1 comply with the actual condition of the human body, and can expand the vagina, in such a way that the vagina is under a uniform pressure, and the treatment effect is better.

Furthermore, in an optional solution of the present embodiment, a design strength of the sponge body 1 is preferably 1.5 kg/cm². In the present embodiment, the design strength of the sponge body 1 is preferably 1.5 kg/cm², so that the breakage of the sponge body will not occur in the vagina, upon the sponge body is pulled out from the vagina through the pulling thread 4 or tweezers, and thereby requirements of clinical use can be met.

Furthermore, in an optional solution of the present embodiment, a breaking elongation of the sponge body 1 is preferably 110%. In the present embodiment, the breaking elongation of the sponge body 1 is preferably 110%, so that the elasticity thereof can meet requirements of clinical use.

Furthermore, in an optional solution of the present embodiment, the sponge body 1 is a medical polyvinyl alcohol sponge sterilized by cobalt-60 irradiation. In the present embodiment, the sponge body 1 is sterilized by cobalt-60 irradiation, so that the sterilization effect is significant.

Furthermore, in an optional solution of the present embodiment, the sponge body 1 is a medical polyvinyl alcohol sponge packaged through double seal. In the present embodiment, the sponge body 1 is packaged through double seal, so that an aseptic condition can be ensured and requirements of storage and clinical use can be met.

### Second Embodiment

In an optional solution of the present embodiment, as shown in FIG. 2, a sponge for gynecological disease treatment and menstrual hygiene provided in the present embodiment includes a sponge body 1. The sponge body 1 is in a cylindrical shape, and a top portion 2 of the sponge body is hemispherical. A surface of the sponge body 1 is provided with spiral grooves 3. The sponge body 1 is a compressed medical polyvinyl alcohol sponge. In the present embodiment, the sponge body 1 is in a cylindrical shape and the top portion 2 thereof is hemispherical, which makes it easy to place the sponge body into the vagina. The sponge body 1 is a compressed medical polyvinyl alcohol sponge. The medical polyvinyl alcohol sponge has extremely good elasticity, can be well positioned at and adapted to a surface of wound and can also well absorb the medicinal liquid, thereby playing a role of local sustained-release delivery. Clinically, the medicinal liquid is injected into the sponge body 1, and then the sponge body with the medicinal liquid is placed against the lesion in the vagina. In this way, the medicine can be stably administered at a fixed point in large dose, and thus, the medicine can directly enter the lesion to kill pathological cells. The sponge for gynecological disease treatment and menstrual hygiene provided in the present embodiment can be also used as a menstrual hygiene product which has a well leakage-proof effect. When the sponge body is taken out, the spiral grooves 3 may carry, solid wastes such as endometrium shedding and blood clots, out from the body.

Furthermore, in an optional solution of the present embodiment, reference is made to first embodiment for other provisions.

### Third Embodiment

In an optional solution of the present embodiment, as shown in FIG. 3, a sponge for gynecological disease treatment and menstrual hygiene provided in the present embodiment includes a sponge body 1. The sponge body 1 is in a cylindrical shape, and a top portion 2 of the sponge body is hemispherical. A surface of the sponge body 1 is provided with hemispherical grooves 3. The sponge body 1 is a compressed medical polyvinyl alcohol sponge. In the present embodiment, clinically, the medicinal liquid is injected into the sponge body 1, and then the sponge body with the medicinal liquid is placed against the lesion in the vagina. In this way, the medicine can be continuously and stably administered, and thus, the medicine can directly enter the lesion to kill pathological cells. The sponge for gynecological disease treatment and menstrual hygiene provided in the present embodiment may be also used as a menstrual hygiene product which has a well leakage-proof effect. When the sponge body is taken out, the hemispherical grooves 3 may carry, solid wastes such as endometrium shedding and blood clots, out from the body.

Furthermore, in an optional solution of the present embodiment, reference is made to first embodiment for other provisions.

### Fourth Embodiment

In an optional solution of the present embodiment, as shown in FIG. 4, a sponge for gynecological disease treatment and menstrual hygiene provided in the present embodiment includes a sponge body 1. The sponge body 1 is in a cylindrical shape, and a top portion 2 of the sponge body is hemispherical. A surface of the sponge body 1 is provided with hook-shaped grooves 3. The sponge body 1 is a compressed medical polyvinyl alcohol sponge. In the present embodiment, the medicinal liquid is injected into the sponge body 1, and then stable administration of a large dosage can be carried out at a fixed point. The sponge for gynecological disease treatment and menstrual hygiene provided in the present embodiment may be also used as a menstrual hygiene product which has a well leakage-proof effect. When the sponge body is taken out, the hook-shaped grooves 3 can carry, solid wastes such as endometrium shedding and blood clots, out from the body.

Furthermore, in an optional solution of the present embodiment, reference is made to first embodiment for other provisions.

### Fifth Embodiment

In an optional solution of the present embodiment, based on the first embodiment, the shape of the grooves can further be such as umbrella-shape and shutter-shape (not shown in figures), which all have the purpose of carrying the solid wastes such as endometrium shedding and blood clots out from the body.

Finally, it is to be explained that the above-mentioned embodiments are merely used to illustrate the technical solutions of the present disclosure, rather than limiting the disclosure. While the detailed description is made to the present disclosure with reference to the above-mentioned embodiments, those ordinarily skilled in the art should understand that modification can still be made to the technical solutions recited in the above-mentioned embodiments, or equivalent substitutions can be made to some or all of the technical features. These modifications or substitutions will not make the corresponding technical solutions essentially depart from the scope of the technical solutions of the embodiments of the present disclosure.

1. A sponge for gynecological disease treatment and menstrual hygiene, comprising a sponge body, wherein the sponge body is in acylindrical shape, a top portion of the sponge body is hemispherical, a surface of the sponge body is provided with annular, spiral, hemispherical or hook-shaped grooves, and the sponge body is a compressed medical polyvinyl alcohol sponge.

2. The sponge for gynecological disease treatment and menstrual hygiene according to claim 1, wherein a coating is provided on a surface of the hemispherical top portion of the sponge body, and the coating is a medical vaseline or antibiotic ointment coating.

3. The sponge for gynecological disease treatment and menstrual hygiene according to claim 2, wherein the coating extends downwards from the hemispherical top portion of the sponge body, and covers ¼ to ½ of a total length of the sponge body.

4. The sponge for gynecological disease treatment and menstrual hygiene according to claim 1, wherein a pulling thread is provided at a bottom of the sponge body.

5. The sponge for gynecological disease treatment and menstrual hygiene according to claim 1, wherein a diameter of the sponge body is 10-20 mm, and a length of the sponge body is 40-80 mm.

6. The sponge for gynecological disease treatment and menstrual hygiene according to claim 5, wherein the diameter of the sponge body after swelling is 20-40 mm, and the length of the sponge body after swelling is 90-130 mm.

7. The sponge for gynecological disease treatment and menstrual hygiene according to claim 1, wherein strength of the sponge body is designed to be greater than 1 kg/cm².

8. The sponge for gynecological disease treatment and menstrual hygiene according to claim 1, wherein a breaking elongation of the sponge body is greater than 100%.

9. The sponge for gynecological disease treatment and menstrual hygiene according to claim 1, wherein the sponge body is a medical polyvinyl alcohol sponge processed by cobalt-60 irradiation sterilization.

10. The sponge for gynecological disease treatment and menstrual hygiene according to claim 1, wherein the sponge body is a medical polyvinyl alcohol sponge packaged through double seal.
